(19) United States
(12) Patent Application Publication
(10) Pub. No.: US 2006/0122606 A1
(43) Pub. Date: Jun. 8, 2006

(54) Radial osteogenic distractor device

(76) Inventor: Philippe Wolgen, Surrey (GB)

Correspondence Address: Bacon & Thomas, 4th Floor, 625 Slaters Lane, Alexandria, VA, 22314-1176, US

(21) Appl. No.: 10/518,587

(22) PCT Filed: Jul. 1, 2002
(86) PCT No.: PCT/EP02/07242

§ 371 Date: Jan. 19, 2006

(51) Int. Cl. A61F 2/30

(52) U.S. Cl. 606/71

(57) ABSTRACT

The invention concerns a radial osteogenic distractor device comprising a stationary member (1) and at least one translating member (2, 2′), the at least one translating member (2; 2′) being slidable within at least one inner lateral channel (10; 21, 22) of the stationary member (1), both the stationary and translating members (1, 2; 2′) being provided with respective fixation holes (3) for fixation to an underlying bone, wherein means (5, 8, 12, 13) are provided at an inner edge of the at least one transtating member (2, 2′) which is opposite to a lateral edge (6) of the at least one translating member (2, 2′) facing the at least one channel (10; 21, 22) of the stationary member (1) to displace the at least one translating member (2, 2′) within the at least one channel (10; 21, 22) in respect to the stationary member (1).

The present invention relates in general to a radial osteogenic distractor device and in particular to a multiplanar radial osteogenic distractor device comprising a stationary member, a translating or distraction member with indentations, a pinion gear matching the indentations of the translating member, wherein the lateral fixation pins of the members do not interfere with the osteogenic site. The latter applies for the entire device. The osteogenic distractor of the present invention can be used in methods to close defects in a concave or convex fashion.

From U.S. Pat. No. 5,700,263 there is known a distraction device for osteogenesis which includes a first member consisting of a curved bar and a second member comprising a housing, the curved bar being telescopically housed in the housing. Both the bar and the housing are secured to respective bone segments by means of bone screws passing through a number of eyelets fixed to the bar and the housing. An extension of the housing includes a ratchet wheel provided with teeth engaging corresponding teeth on the bar, such that the rotation of the ratchet wheel causes the translation of the bar within the housing. A tension mechanism is further provided for firmly pressing guide rollers against the bar.

The device according to U.S. Pat. No. 5,700,263 is, however, bulky and does not allow a accurate guiding of the bar within the housing. In addition, the transmission of the force to the bar for the generation of the translational movement of the bar within the housing is not free of shortcomings.

Therefore, it is an object of the present invention to provide for a radial osteogenic distractor device which eliminates the drawbacks known from the prior art.

A particular object of the present invention is the provision of a radial osteogenic distractor device which provides for a satisfactory transmission of the force to a translating member within a stationary member, such as to ensure a smooth gliding operation of the translating member within the stationary member.

A further object of the present invention is the provision of a radial osteogenic compact distractor device with a safe guidance for a translating member within a stationary member.

The above and further objects which will become apparent hereinafter are achieved by a radial osteogenic distractor device as defined in the appended claims.

The invention is herein described, by way of example only, with reference to the accompanying drawings, wherein:

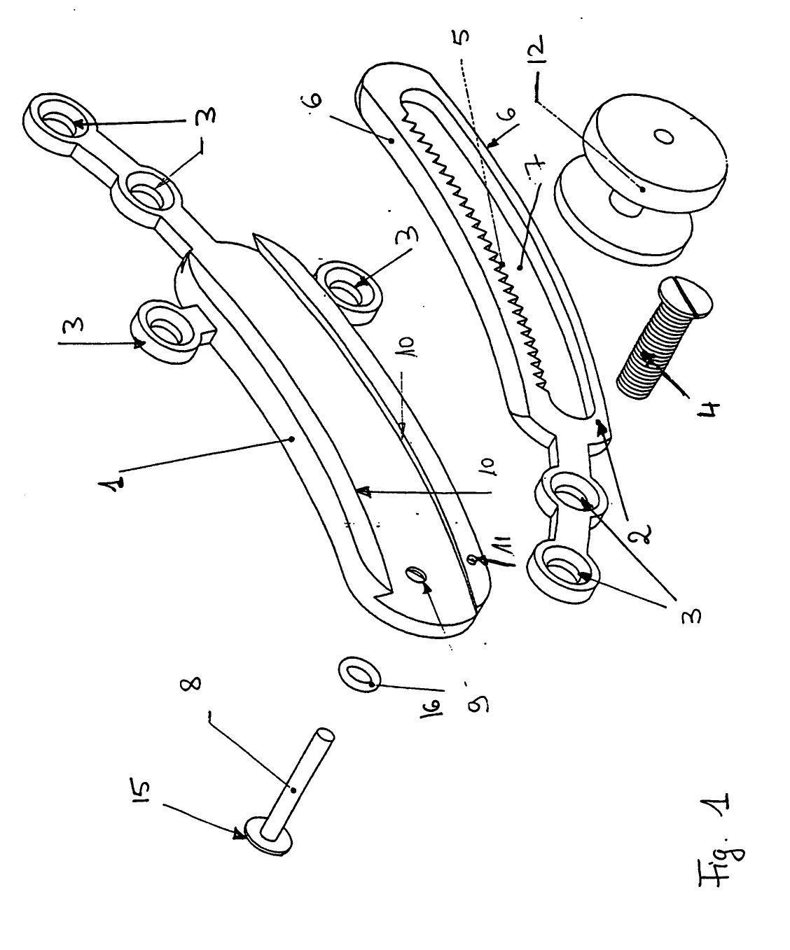

FIG. 1 represents an exploded view of a cranio-facial radial osteogenic distractor device according to a first preferred embodiment of the present invention;

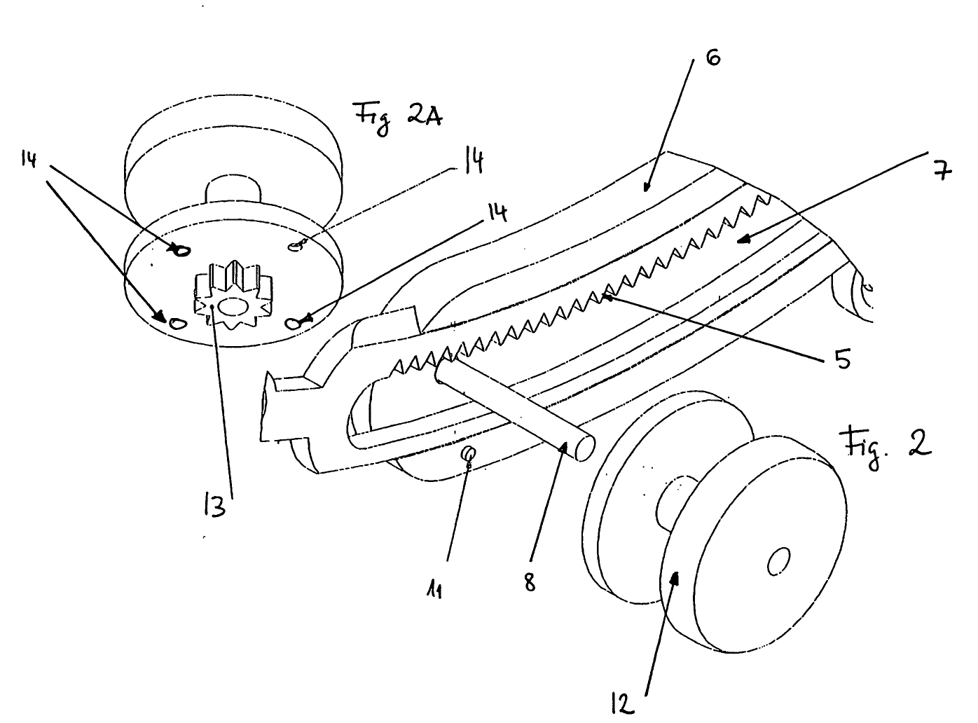

FIGS. 2 and 2A represent an enlarged view of the driving mechanism for the translating member;

FIG. 3 shows the radial osteogenic distractor device of FIG. 1 in an assembled state;

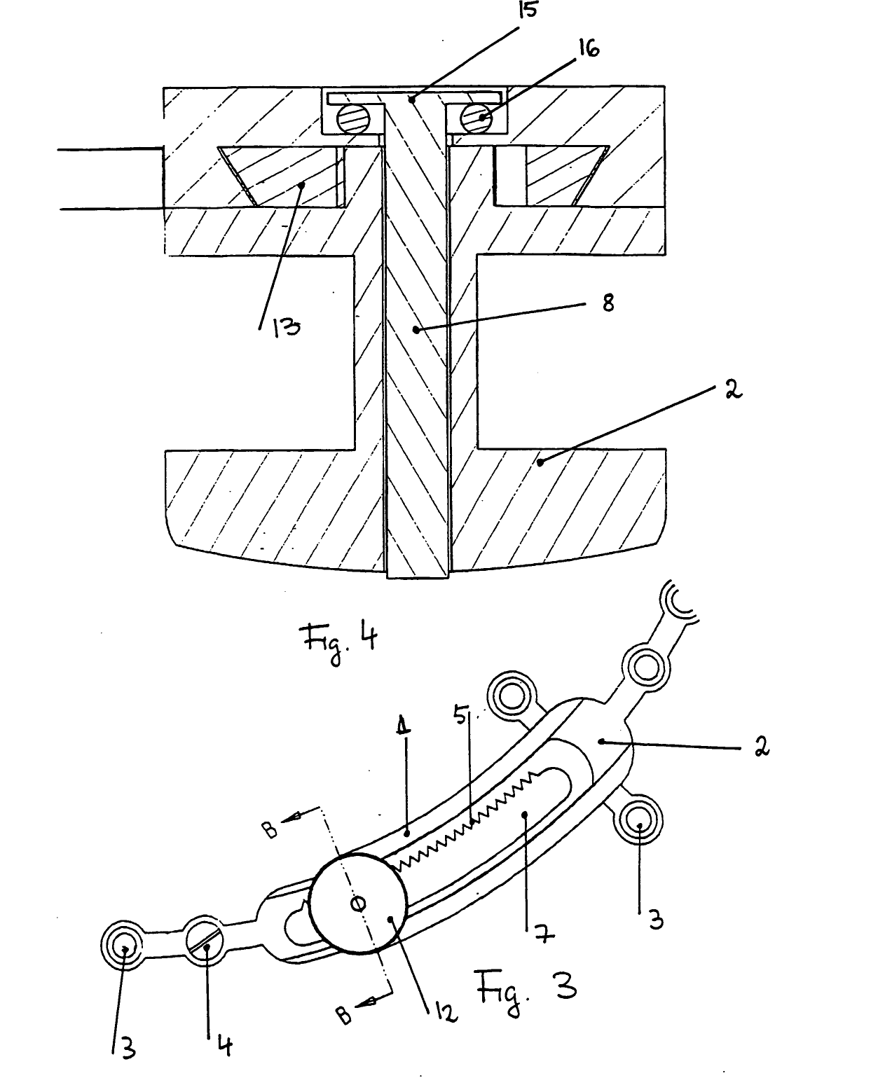

FIG. 4 shows a sectional view of the radial osteogenic distractor device according to the first preferred embodiment of the invention taken along line B-B of FIG. 3;

FIG. 5 shows a perspective view of the radial osteogenic distractor device of FIG. 1 in an assembled state;

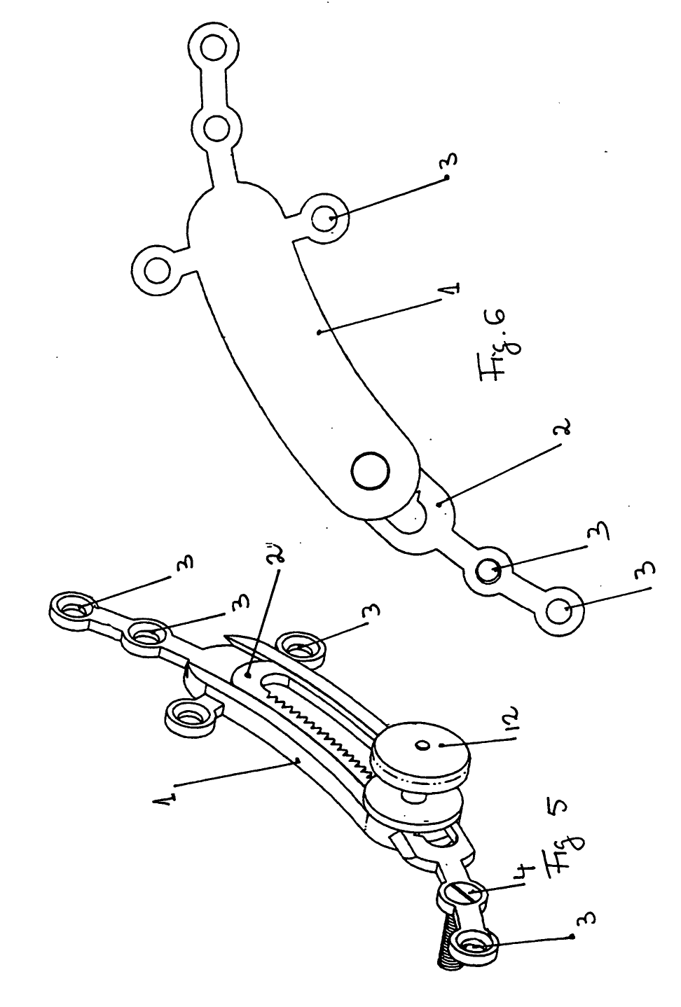

FIG. 6 shows a bottom view of the radial osteogenic distractor device of FIG. 1 in an assembled state;

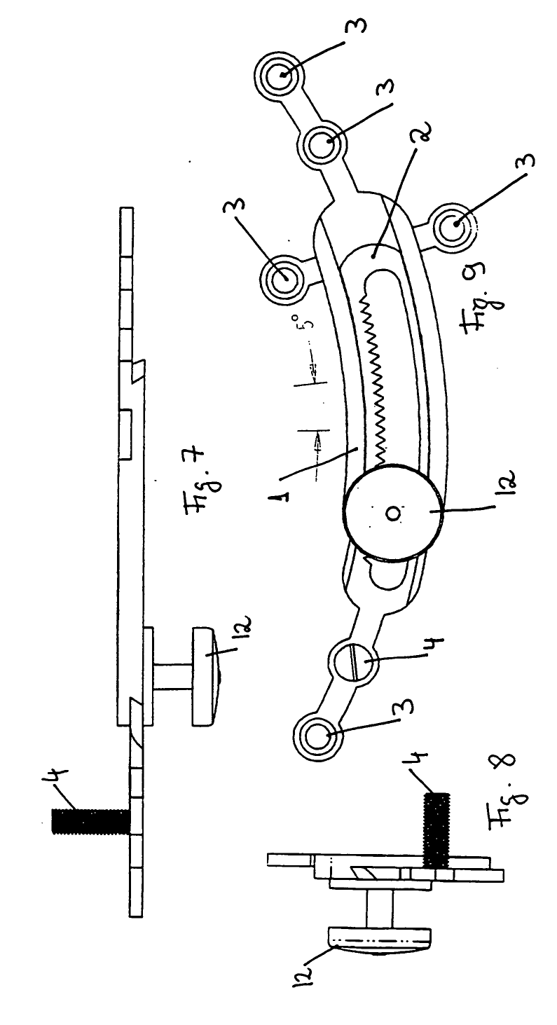

FIGS. 7 and 8 represent side views of the radial osteogenic distractor device of FIG. 1 in an assembled state;

FIG. 9 shows a top view of the radial osteogenic distractor device of FIG. 1 in an assembled state;

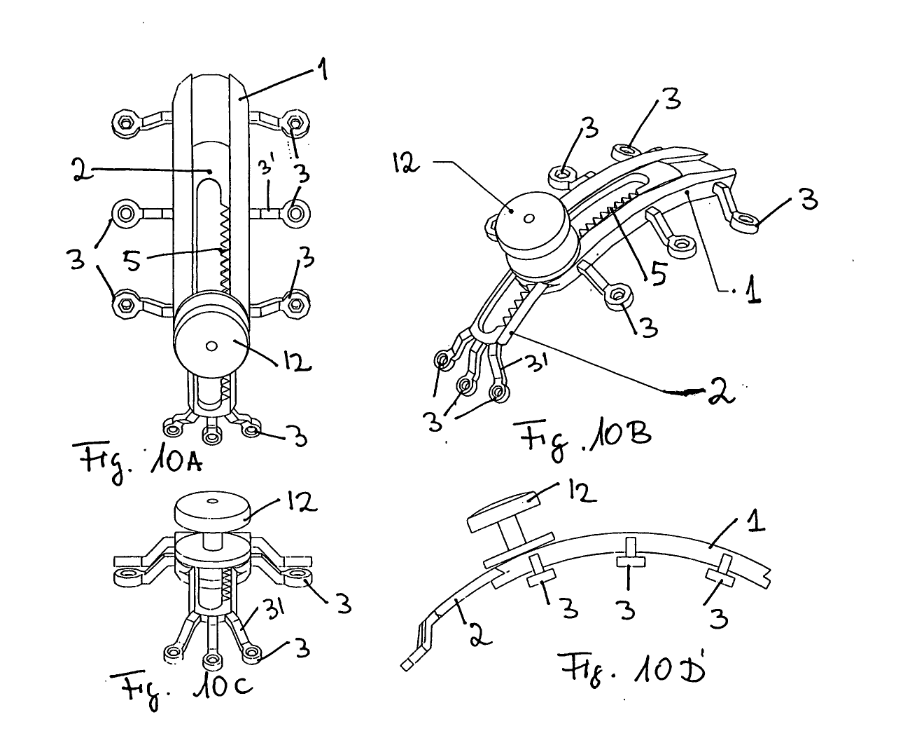

FIGS. 10A through 10D represent various views of a cranial radial osteogenic distractor device according to a second preferred embodiment of the present invention;

FIG. 11 represents an exploded view of a radial osteogenic distractor device according to a third preferred embodiment of the present invention devised as a bifocal “mandibular and maxilary” distractor;

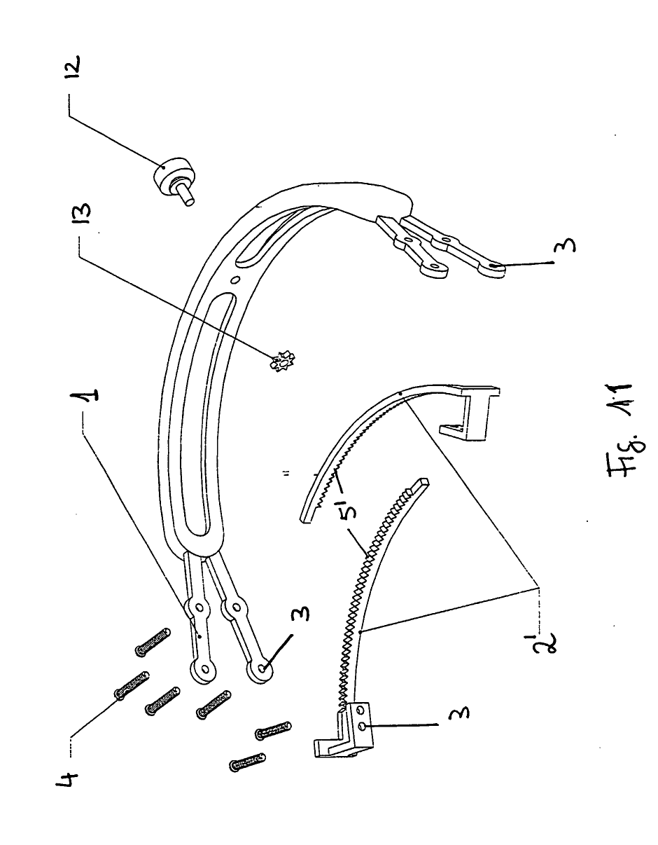

FIG. 12 shows a rear view of the radial osteogenic distractor device according to the third preferred embodiment of the present invention in an assembled state;

FIG. 13 shows a sectional view along line A-A of FIG. 11;

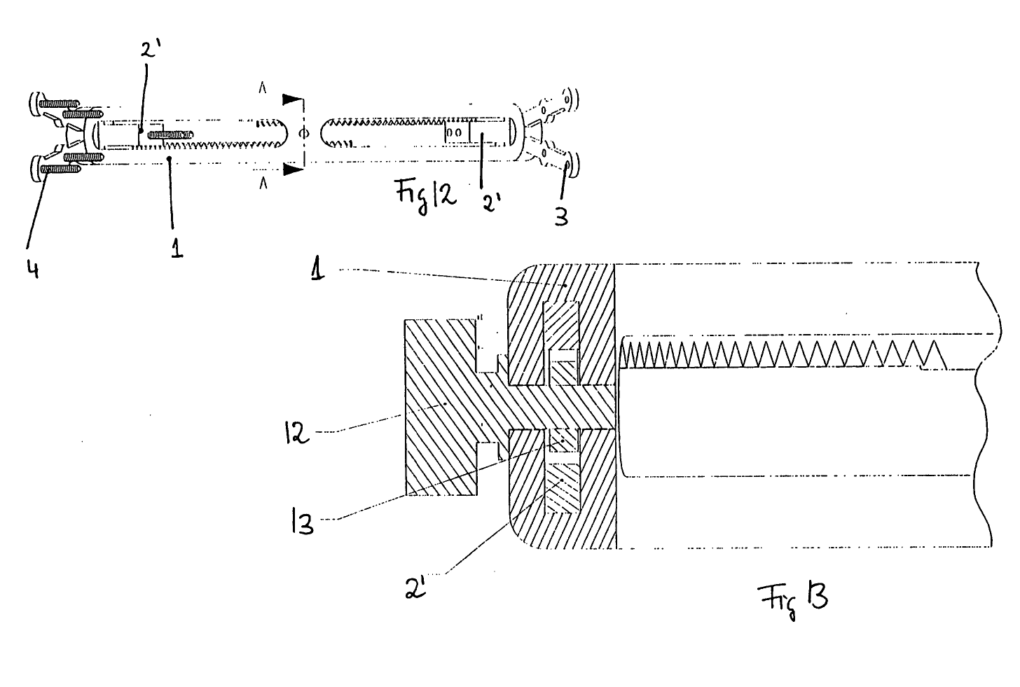

FIGS. 14 and 15 show perspective views of the distractor device of FIG. 11;

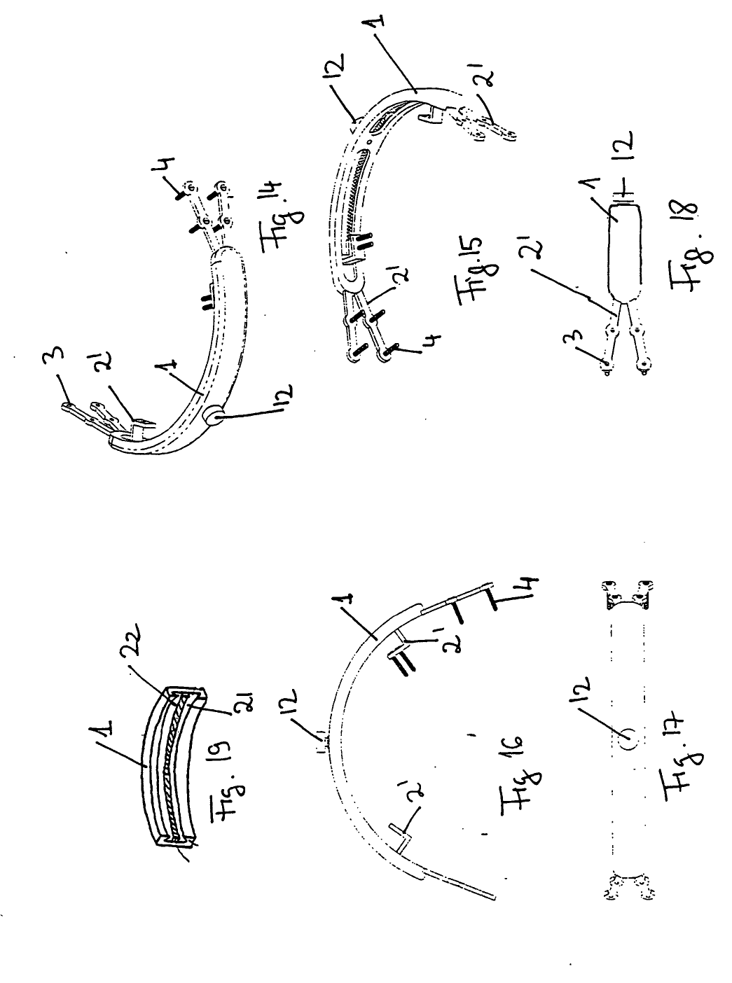

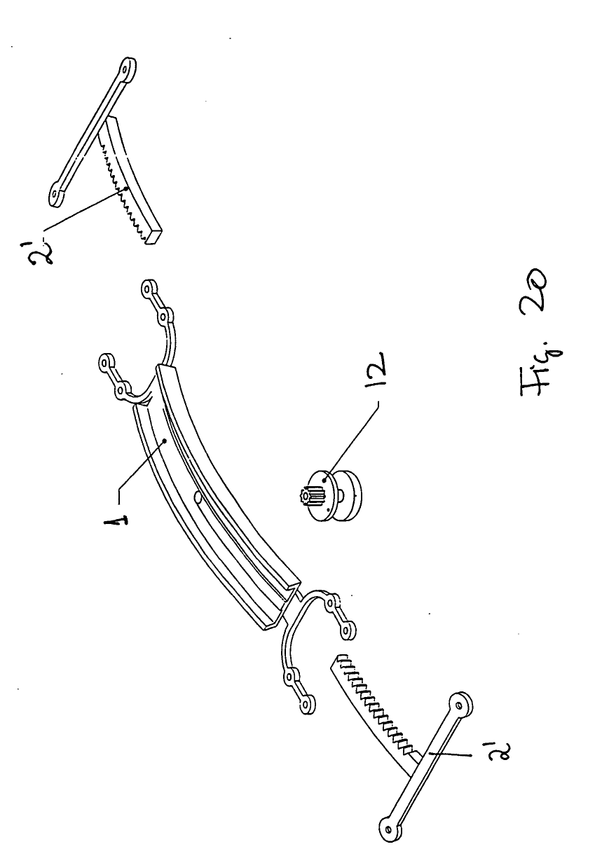

FIGS. 16 through 18 show upper, front and lateral views, respectively, of the distractor device of FIG. 11;

FIG. 19 shows a detailed view of the channels provided in the stationary member for slidingly accommodating the multiple translating members in the third preferred embodiment of the present invention;

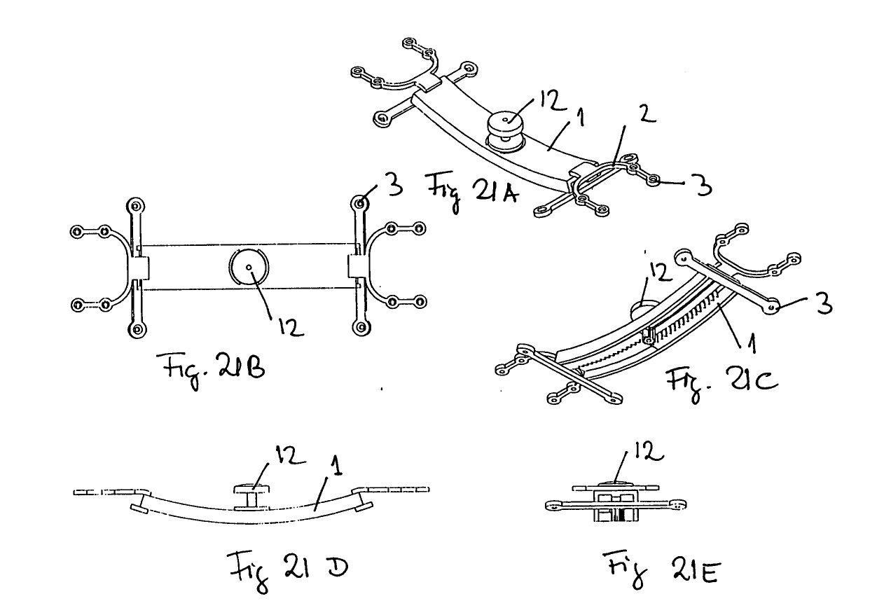

FIG. 20 represents an exploded view of a radial osteogenic distractor device according to a fourth preferred embodiment of the present invention devised as a cleft “intra-oral” distractor device; and

FIGS. 21A through 21E represent various views of the cleft distractor device according to the fourth embodiment of the present invention.

With reference to FIG. 1 there is shown a cranio-facial radial osteogenic distractor device, wherein the device is smooth and without sharp edges. Preferably the device according to the invention relates to a device which transports one or more bone fragments.

The distractor device includes a stationary member 1 and a translating member 2 which is slidably received by the stationary member 1. Both the stationary and the translating members 1-2 are provided with respective fixation holes which are designated with reference numeral 3. As shown, the fixation holes 3 extend on the lateral, cranial, caudal side of a not shown underlying bone in a sagittal, transversal or vertical plane. The surface of the fixation holes 3 is smooth and unthreaded. The fixation holes 3 are embedded in the surface of the distraction device.

Each fixation hole 3 is adapted for accommodating a respective fixation pin 4. For the sake of simplicity only one of the fixation pins is shown. The pins are intended for the fixation of the translating and stationary members 1-2 to the underlying bone in a manner known to the person skilled in the art.

The translating or distraction member 2 shown in FIGS. 1 and 2 has preferably a curvilinear shape, although other shapes like rectilinear could be envisaged.

The translating member 2 preferably comprises a recess 7. Preferably the recess 7 is parallel to the inner surface of the stationary member 1, the recess 7 comprising indentations 5 on one side in the transversal, vertical or sagittal plane. Preferably the indentations 5 are located at the antagonistic (opposite) side of the concave or convex or curvilinear surface of the translating member 2 defining the recess 7. The indentations 5 are not in contact with the underlying osseous (bony) surface and do not interfere with the osteogenic site. Length, width and thickness of the recess 7 are chosen, as known to the person skilled in the art, according to the specific dimensions and requirements of the osteogenesis site.

Perpendicular to the translating member 2, towards the bony surface (in a sagittal, transversal or vertical plane), the translating member 2 includes a retaining shaft 8 which may be integral with the translating member 2. Alternatively the retaining shaft 8 fits into an opening 9 of the stationary member 1 and can be inserted at the beginning of the recess 7 of the translating member 2. The retaining shaft 8 has a collar or head 15 which restricts its movement through the opening 9 of the stationary member 1.

According to the invention the lateral edges 6 of the translating member 2 are beveled and fit into inner lateral channels 10 of the stationary member 1. The surface of the translating member 2 facing in the assembled state the stationary member 1 is congruent with the inner surface of the stationary member 1. Thus, the inner surface of the stationary member 1 and the inner lateral channels 10 thereof define a configuration providing for the sliding of the translating member 2 into the stationary member 1 in a sagittal, transversal or vertical plane. In a preferred aspect of the invention the inner lateral channels 10 may be advantageously shaped in a dovetail configuration.

The stationary member 1 includes, as explained above, the fixation holes 3 for the securing thereof on the lateral, cranial, caudal side of the underlying bone in a sagittal, transversal or vertical plane. As described, the surface of the fixation holes 3 is smooth and unthreaded. The fixation holes 3 are embedded in the surface of the stationary member 1.

The stationary member 1 furthermore includes at least one stationary pin 11 positioned perpendicularly to and integral with the stationary member 1. The stationary pin 11 is round and smooth and non-threaded. The stationary pin 11 fits into the lower side of the four openings 14 provided on an actuator knob 12. By engaging the stationary pin 11 with one of the openings 14 on the actuator knob 12 it is possible to firmly stop the actuator knob 12 in a number of predefined positions. The number of the opening 14 may vary according to the required positioning precision.

According to a central aspect of the invention there is provided, as shown in FIG. 2A, a pinion gear 13 on the knob 12 for interaction with the indentations 5 of the recess 7 of the translating member 2.

The top surface and edges of the pinion gear 13 are smooth. The pinion gear 13 fits into the actuator knob 12. The top surface of the pinion gear 13 includes a radial groove.

The actuator knob 12 is a hollow cylinder, round in diameter and its lateral surface is beveled. Its lower surface (in a sagittal, vertical or transversal plane) contains the four openings 14 which are preferably ellipse shaped. The inner surface of the actuator knob 12 has the shape of a cylinder and is non-threaded.

The top surface of the actuator knob 12 is rounded off, smooth and contains a (not shown) metric radial indicator and depicts an arrow to indicate the direction of rotation and activation.

As best seen in FIG. 4, in the assembled state of the radial osteogenic distractor device according to the invention, a spring detent 16 is cooperating with the retaining shaft 8 to hold the actuator knob 12 when the retaining shaft 8 is threadingly engaged with the actuator knob 12. The spring detent 16 is preferably in the shape of an O-ring or a washer. It can also be embodied as an actual spring or a coil of three or four windings, the latter embodiments not being shown.

The retaining shaft 8 is threaded, although no threads are shown, so as to be threadingly received by the actuator knob 12. The head of the shaft is beveled and is continuous with the upper part of the actuator knob 12 when the device is in a non-activated mode. The head of the shaft contains a groove over the entire diameter of the upper surface. The apical or lower ⅓part of the shaft is threaded and has a rounded converging tip.

Preferably, the members 1-2, the pinion gear 13, the spring detent 16, the actuator knob 12, the retaining shaft 8, the retaining collar 15 and the fixation pins 4 are made of biocompatible materials such as metals, polymers, and mixtures thereof. Preferred examples are metals (stainless surgical steel, Titanium), metal alloys (Nickel-Titanium, Cobalt-Chromium alloys, Cobalt-Chromium-Nickel, CrCoMo), and polylactides. The most preferred example is Titanium. Titanium has extraordinary tissue compatibility. In order to improve the rigidity of the device, each of the members is preferably made of one piece of material. Preferably, the material and edges of the entire device are smooth without sharp edges.

When the radial osteogenic distractor device according to the present invention is assembled there is provided the stationary member 1, the translating member 2 with indentations 5, the pinion gear 13, the spring detent 16, the actuator knob 12, the retaining shaft 8 and the retaining collar 15, wherein the translating member 2 slides through the underlying stationary member 1 and where the actuator knob 12 is turned in order to activate the device. The present invention further provides for a method of multiplanar radial osteogenic distraction of two or more bone pieces using the device comprising the stationary member 1 and a translating member 2 with a pinion gear 13, the spring detent 16, the actuator knob 12, the retaining shaft 8, and the retaining collar 15, where the stationary member 1 and the translating member 2 are fixed to the underlying bone by the lateral osteosynthetic fixation pins 4. Hereby the pinion gear 13 is inserted into and rotates along the recess 7 of the translating member 2 and the inner surface of the stationary member 1. The spring detent 16 is mounted near the top of the pinion gear 13, and both the spring detent 16 and the pinion gear 13 fit within the actuator knob 12 in a (sagittal, transversal or vertical) plane away from the stationary and translating members 1-2. The two pieces or bones are distracted by turning the actuator knob 12. The device does not interfere with the neo-osteogenic site.

As apparent to the person skilled in the art, the dimensions of the present device may be suitable and tailored in some practical applications of the present invention to distraction histio-osteogenesis in the mandible and maxilla and the cranial bones and the tibia and femur and the radius and ulnus and humerus. With the design of the device an attempt is made to provide for all parameters affecting distraction osteogenesis, for instance in patients with retrognathia of the mandible and or maxilla or patients with cranial defects. The device may also be used in the edentulous mandible and/or maxilla, and dentulous (tooth bearing) patients with alveolar defects. The simplicity and the size of the device make it easily applicable and more suited in the craniofacial skeleton and oral cavity and for use in the long pipe bones. The device of the present invention has further advantages over the prior art. The device is small. Its small size allows for application in the orofacial skeleton and allows implantation underneath the mucoperiosteal layer. The small size further leaves little scar tissue after distraction. In the long pipebones the device holds further advantage over the prior art by its simplicity and its unifocal penetration by the actuator knob through the mucoperiosteal layers and skin. In the craniofacial skeleton and the long pipebones the device enables the patient to observe the rate of distraction and also to hear the distraction distance by its ‘clicking’ sound coinciding with one unit of distraction over the intended distance to be distracted.

In the orofacial skeleton the design of the device allows for placement and removal through a simple surgical procedure, without the requirement for general anesthesia of the patient. The size and design of the device presented enables the surgeon to use a minimally invasive surgical approach when placing the device. The design avoids interference with the site of neo-osteogenesis. The device is further designed such that it produces little or no inconvenience or irritation for the patient during the period of distraction. Furthermore, in a preferred embodiment of the device of the invention the design of the device makes the chance of infections and other complications particularly small. As a consequence of the design, the device has high rigidity, potentially leading to fewer complications than the devices of the prior art. The design avoids micro movements at the site of neo-osteogenesis.

A further advantage of the device lies in its multiplanar transport. This provides for particular advantages, inasmuch as it is preferable to manufacture a convex or a concave or a curvilinear device for the two-dimensional distraction.

The activation of the device takes place by turning the actuator knob according to the indicator on the actuator knob and by adhering to the ‘clicking’ sound of the actuator knob corresponding to a given distance of transport.

The design of the device according to the present invention accommodates for the specific features of the tight periosteal, mucoperiosteal or dermal layer. In order to protect the periosteal or mucoperiosteal layer under which the device is to be inserted the present invention provides for the following advantages:

a) the edges of the brackets are preferably rounded/curved;

b) the transition from the stationary member to the translating member is preferably smooth and rounded off;

c) the sliding movement of the translating member along and within the stationary member is not causing any damage to the overlying soft surfaces;

d) the proposed titanium or any material used is preferably highly polished in order to avoid bacterial accumulation or bacterial vegetation at the surface of the material; and

e) the top part of the actuator knob when using in the orofacial skeleton is preferably rounded off and may consist of a soft polymer or latex or soft acrylic.

The positioning of the parts of the distractor is as follows:

Step 1: The device needs to be fixated onto the cortical surface of the stationary segment of the underlying bone (in a sagittal, vertical or sagittal plane) by screwing the fixation pins 4 into the fixation holes 3 of the stationary member 1.

Step 2: The translating member 2 needs to be positioned over the stationary member 1 (in a sagittal, transversal or vertical plane). Hereafter the pinion gear 13 may be inserted into both members 1-2.

Step 3: The spring detent 16 needs to be inserted into the actuator knob 12 and consequently the actuator knob 12 needs to be inserted over the pinion gear 13.

Step 4: The retaining shaft 8 needs to be inserted into the knob 12 and manually screwed with a designed screwdriver until resistance is felt.

Step 5: The translating member 2 needs to be fixated by the fixation pins 4 onto the cortical surface of the bony segment to be transported.

The device of the present invention is preferably manufactured by preparing the stationary member as the base member and the translating member as the member fitting into or onto the stationary member. The pinion gear, spring detent and actuator knob need to be manufactured in matching dimensions and mounted into or onto the stationary and the translating members. Turning the actuator knob as indicated will activate the device. Preferably the parts of the device of the present invention are manufactured without using welding or soldered connections. Instead each part is preferably manufactured out of one solid (integral) piece through casting.

Therefore, preferably the stationary member and the translating member are manufactured out of one piece, preferably the pinion gear, spring detent and actuator knob being also made out of one piece.

The distraction process is as follows. When turning the actuator knob in the direction of the indicated arrow on top of the actuator knob the device is activated. The distance of activation is to be noticed by the audible detent of the device. Distraction is then performed according to the clicks heard and as instructed by the treating physician. By gently lifting the actuator knob it is possible to reverse the distraction distance, i.e. against the direction as indicated by the arrow on top of the actuator knob. At the end of the distraction period the distractor is left in situ. During the consolidation phase, one may decide to remove the retaining shaft 8 out of the actuator knob and remove hereafter the actuator knob and the retaining shaft 8 by counter clockwise rotation. The stationary member and translating member may stay in situ during the consolidation phase or as long as deemed necessary. Transport of the translating member and transported segment is along the curvature of the recess of the member and the recess of the translating member. The end of distraction is reached when one wishes to terminate the distraction process or when the end of the recess of the stationary member has come in contact with the pinion gear.

The practical use of the device according to the present invention is exemplified below:

### EXAMPLE 1

First a mucoperiosteal incision is performed along the body of the mandible and ascending ramus of the mandible. After careful reflection of the mucoperiosteal layers the buccal mandibular bone is exposed. Then a sagittal split osteotomy is being performed bilaterally and the buccal and lingual cortex are being split and separated. With respect to the inferior alveolar nerve the mandibular segment is then gently manipulated and the pterygomasseteric sling is stretched in order to give the mandible more freedom of movement.

The device is then positioned over the buccal mandibular surface. The size and angulation of the device are assessed according to the formula as introduced by Wolgen et al. (published) which is hereby incorporated by reference. First the stationary member is positioned and fixed to the underlying proximal mandibular segment. Second the translating member is positioned over—or in another embodiment—slid into the stationary member and the pinion gear, spring detent and actuator knob are positioned into the recess of the stationary and translating member. Finally the retaining shaft 8 is inserted into the actuator knob. When this is performed bilaterally the device may be activated. The transported segment of the mandible follows a curved trajectory of distraction. The end of distraction is reached when the anterior mandibular teeth meet the maxillary anterior antagonists in an end-to-end relation.

### EXAMPLE 2

First a mucoperiosteal incision is performed along the body of the mandible and ascending ramus of the mandible. After careful reflection of the mucoperiosteal layers the buccal mandibular bone is exposed. The mucoperiosteal layers on the lingual side of the mandible are exposed and carefully reflected. Hereafter coming from the buccal side a mandibular bone hook is inserted underneath the mandibular border. With a microsaw a corticotomy of the buccal mandible is performed. The same is performed on the lingual cortex of the mandible. With a round bur the inferior border of the mandible is osteotomized. Careful separation of the distal and proximal mandibular segments are performed, without mobilizing the distal segment. Hereafter the device is placed and fixed on the buccal side of the mandible. According to the pre-operative planning the device is placed and its path of distraction is carefully assessed.

In a variation of the first preferred embodiment of the present invention which is shown in FIGS. 10A through 10D there is provided a cranial distractor. The cranial distractor of the second preferred embodiment basically differentiates from the first preferred embodiment in that the bent radiuses of the edges of the stationary and translating member 1, 2 is substantially equal. Furthermore, the fixations holes 3 are spaced from the members 1, 2 by means of legs 3′.

In a third preferred embodiment according to the present invention which is shown in FIGS. 11 through 19 multiple translating members 2′ are used in order to transport bony segments in different directions in a bifocal “mandibular and maxilary” distractor. Except for the differences explained hereinbelow, the third preferred embodiment of the invention is similar to the first one, and therefore detailed explanations in respect to elements which are the same in both embodiments will be omitted. In a further variation of the present invention the distractor can be embodied as a multifocal device, for instance a quatrofocal device wherein each of the translating members shown in FIGS. 11 through 19 is provided with a respective separate knob.

The number of the translating members is, as shown in FIG. 11, preferably two. In this embodiment the translating members 2′ slide within the stationary member 1 in opposite directions activated by one pinion gear 13, one spring detent (not shown) and one actuator knob 12. In this embodiment the end of distraction is reached when the translating members 2′ have come to the end of the recess of the stationary member 1. As shown in FIG. 11 the respective indentation 5′ of the translating members 2′ overlap at the central region of the stationary member 1, such that a single actuator knob with its pinion gear may be used to activate the translating members. With this device distraction is performed with a rate of 0.1 to 1.0 mm per day.

The translating members 2′ are slidingly accommodated in respective channels 21 and 22 of the stationary member 1. Preferably, the channels are dovetail shaped and extend through the entire length of the stationary member 1.

A variation of the third preferred embodiment of the present invention with two translating members 2′ is shown in FIGS. 20 and 21A through 21E. In a similar manner to the second embodiment of the invention the respective bent radiuses of the translating and stationary members are substantially equal.

Where technical features mentioned in any claim are followed by reference signs, those reference signs have been included just for the sole purpose of increasing intelligibility of the claims and accordingly, such reference signs do not have any limiting effect on the scope of each element identified by way of example by such reference signs.

1. A radial osteogenic distractor device comprising a stationary member and at least one translating member, the at least one translating member being slidable within at least one inner lateral channel of the stationary member, both the stationary and translating members being provided with respective fixation holes for fixation to an underlying bone, wherein means are provided at an inner edge of the at least one translating member which is opposite to a lateral edge of the at least one translating member facing the at least one channel of the stationary member to displace the at least one translating member within the at least one channel in respect to the stationary member.

2. The device according to claim 1 wherein the displacing means comprises a pinion gear provided on a retaining shaft attached to the stationary member and extending through a recess of the translating member to engage respective indentation provided at the inner edge of the at least one translating member.

3. The device according to claim 1 wherein the translating member is formed of a single member, the single member being of curvilinear shape, the single member including lateral edges which are shaped to fit into the curvilinear inner lateral channels of the stationary member.

4. The device according to claim 3 wherein the indentation provided at one inner edge only of the recess.

5. The device according to claim 1 wherein the translating member is formed of two members, each member having respective indentations at the inner edge thereof, each member including a respective lateral edge which is shaped to fit into a respective inner lateral channel of the stationary member.

6. The device according to claim 2, wherein the displacing means further include an actuator knob to which the pinion gear is attached.

7. The device according to claim 6 wherein the actuator knob is provided with at least one opening which is sized to accommodate a stationary pin positioned perpendicularly to and integral with the stationary member, such as to lock the actuator knob in a fixed position.

8. The device according to claim 7 wherein the retaining shaft has a collar or head which restricts its movement through an opening of the stationary member and wherein a spring detent is interposed between the head and the stationary member.

9. The device according to claim 8 wherein the spring detent is devised to cooperate with the retaining shaft to hold the actuator knob when the retaining shaft is threadingly engaged with the actuator knob and wherein the spring detent is preferably in the shape of an O-ring or a washer.

10. The device according to claim 1 wherein the channels are dovetail shaped and extend through the entire length of the of the stationary member.

11. The device according to claim 1 wherein the displacing means comprises a pinion gear provided on a retaining shaft attached to the stationary member and extending through a recess of the translating member to engage respective indentation provided at the inner edge of the at least one translating member, and wherein the translating member is formed of a single member, the single member being of curvilinear shape, the single member including lateral edges which are shaped to fit into the curvilinear inner lateral channels of the stationary member.

12. The device according to 11 wherein the displacing means further include an actuator knob to which the pinion gear is attached.
